(19) United States
(12) Patent Application Publication
(10) Pub. No.: US 2020/0117833 A1
(43) Pub. Date: Apr. 16, 2020

(54) LONGITUDINAL DATA DE-IDENTIFICATION

(71) Applicant: KONINKLIJKE PHILIPS N.V., ENDHOVEN (NL)

(72) Inventor: Daniel PLETEA, Eindhoven (NL)

(21) Appl. No.: 16/583,357

(22) Filed: Sep. 26, 2019

Related U.S. Application Data
(60) Provisional application No. 62/743,601, filed on Oct. 10, 2018.
(60) Provisional application No. 62/876,082, filed on Jul. 19, 2019.

(51) Int. Cl. G06F 21/62; G16H 10/60; G16H 70/60

(52) U.S. Cl. CPC G06F 21/6254; G16H 70/60; G16H 10/60

(57) ABSTRACT

A system and method for anonymization of a data set of patient data from multiple patients provides k-anonymity, a concatenation of indirect identifiers of a patient enabling identifying an outlying patient in the data set if there are less than k patients having a same concatenation of indirect identifiers. The patient data as provided (302) is longitudinal and has events related to a disease or a treatment of a disease, and time stamps related to the events. At least one first indirect identifier representing a property of the data distribution of the time stamps, and at least one second indirect identifier representing a number of events regarding a respective patient, are determined (303). For all patients in the data set, the respective concatenations comprising the first indirect identifier and the second indirect identifier, are determined (305). Then, the patient data of each outlying patient is removed from the data set (306).

### FIELD OF THE INVENTION

The present invention relates to the analysis of the handling of personally identifiable information (PII), such as patient data. More specifically, the present invention relates to the analysis and de-identification of patient data with respect to sequences of events related to a disease or treatment, such sequences containing time stamps or time related data and being called longitudinal data.

### BACKGROUND OF THE INVENTION

Nowadays medical and health records of patients are collected and used for clinical bioinformatics research. Next to clinical data, imaging data or biobanking data of patients also their patient data are collected, and analyzing patient data plays a significant role in medical research and in diagnostics and anamnesis. For example, the patient data are analyzed for finding or improving treatments for different diseases.

However, analysis of patient data might pose threats for the patients that are sharing their patient data in that, for example, their privacy might be violated. The violation is due to the fact that the patient data of a person may contain personally identifiable information (PII) such as direct identifiers (e.g. name, email address, social security number, medical record number) and indirect identifiers such as locations, gender, age, weight, height eye color, skin color. The longitudinal patient data, e.g. containing time stamps and events, possibly together with other data embedded in the patient data, may lead to identification of a person by analyzing the patient data. In order to protect the privacy of individuals, certain parts of the patient data need to be anonymized when the patient data are provided for medical bioinformatics research and analysis.

Recent regulations, e.g. GDPR (see [1]), HIPAA (see [5]), put very strict requirements on the handling of personally identifiable information (PII), while also putting huge fines on noncompliance. For instance, the GDPR requires a data controller to ask for explicit consent from all data subjects. This consent must be minimal, meaning that a data controller cannot ask for more permissions than the bare minimum necessary. This is especially inconvenient in the context of medical research, where huge amounts of medical data get combined and analyzed in many different ways in the hope of getting new insights. Getting consent from every single data subject for every single analysis is practically impossible.

Luckily, these regulations provide a way out: when the dataset does not contain PII, then the regulations do not apply. Thus, making sure all PII identifiers are removed from the data makes it a lot easier to work with the resulting dataset. This is a commonly used process called anonymization.

The easiest way to remove personal identifiable information (PII) from a dataset seems to be to just remove direct identifiers like names and birthdates, which may be done initially. However, PII can be defined as “any data that could potentially identify a specific individual”. As it turns out, this is much more than just direct identifiers. As an example: an ethnicity of ‘Asian’ may reveal no information when talking about people in an city in China, but can really stand out when talking about a small village in the Netherlands with only one inhabitant of Asian descent. Such potentially sensitive information whose release must be controlled are called quasi-identifiers or indirect identifiers.

Samarati and Sweeney [4] first studied this issue and came up with the concept of k-anonymity which commonly used metric is an example of an anonymity property. Other anonymity measures may also be considered, as elucidated further on. For some predefined value k the k-anonymity property requires that each release of data must be such that every combination of values of quasi-identifiers can be indistinctly matched to at least k individuals. So, the anonymity property defines that a concatenation of all indirect identifiers of a patient enables identifying an outlying patient in the data set if there are less than the predefined value k patients having a same concatenation of indirect identifiers.

Longitudinal data is complex health data that contains information about patients over periods of time, e.g. as depicted in FIG. 1. A person's medical history may be taken as a series of events: when a person was first diagnosed with a disease, when the person received treatment, when the person was admitted to an emergency department, etc. Applying anonymity on longitudinal data is rather difficult. For example, the health data may contain multiple sources of re-identification, e.g.:
dates: date of service, when drugs were dispensed or when specimens were collected.
events: diseases, procedures etc. (e.g. coded by ICD codes, CPT codes).

### SUMMARY OF THE INVENTION

Some of the existing methods for anonymization in bioinformatics research attempt to achieve de-identification of longitudinal timestamps by adding noise. However, this removes the temporal relation between consecutive timestamps and therefore may lead to wrong results during the analysis of this data. In order de-identify longitudinal data the next methods may be used: randomizing dates independently of one another, shifting the sequence while ignoring the intervals, generalizing intervals while maintaining order, see [2].

Shifting dates with keeping intervals intact is considered not safe due to preserving the intervals between consecutive events. This is true when the number of the events is limited but usually this is not the case in longitudinal data, where multiple timestamps and events are attached to the patients. Furthermore randomizing these timestamps at de-identification is not done in a structured manner and may affect the research results.

Attributes of longitudinal may be part of the data, as discussed in [3]. Examples are: length of stay in hospital, number of days since first claim computed from the first claim for that patient for each year, etc. These attributes may be indirect identifiers but are not completely describing the longitudinal record of a patient.

Furthermore, for the events attached to the timestamps the state of the art considers the number of events as an indirect identifier and truncates these events so that each bin of events has the required k-anonymity property. FIG. 2 shows an example of a frequency table for the number of events. In this example the bin with patients which number of events in the range [26 to 30] has the size 4. If k=5 for achieving 5-anonymity, then these patients may be combined with the [21 to 25] bin which may be achieved by truncating some of the events. However, the truncating does not take care of outlying events from the rest of the bins (e.g. rare events). Furthermore this method of truncating events may lead to wrong research results.

According to the foregoing, the prior art has following issues:
De-identification of longitudinal timestamps is done usually by adding noise, as presented in the previous section. This removes the temporal relation between consecutive timestamps and therefore leads to wrong results during the analysis of this data.
Randomly adding noise in the timestamps is not structured enough in order to remove all the outliers. There may be patients who have a medical historic very long, for example more than 20 years, and therefore these outliers would remain in the de-identified dataset.
Rare events in the longitudinal data are outlying the patients even when the patient has many events attached to his longitudinal record. It is necessary for these outliers to be treated during the de-identification process.
Inserting noise by truncation of events is modifying the data in such a manner that may affect the research.
The number of events is not the only indirect identifier from a series of events attached to the longitudinal record of a patient.

It is an object of the invention to provide a method and system for longitudinal data de-identification that takes into account at least one of the preceding issues.

For this purpose, devices and methods for anonymization of a data set of patient data are provided as defined in the appended claims. According to an aspect of the invention a method for anonymization of a data set of patient data from multiple patients for providing a predefined anonymity property is provided as defined in claim 1. A system is provided as defined in claim 14. According to a further aspect of the invention there is provided a computer program product downloadable from a network and/or stored on a computer-readable medium and/or microprocessor-executable medium, the product comprising program code instructions for implementing the above method when executed on a computer.

Advantageously, the method and system achieve that a data set of patient data, in particular longitudinal patient data, is anonymized to a predetermined level as defined by the anonymity property. The relevance of the data set is kept high by only removing outlying patients, while avoiding noise and generalizing of time relate data.

Various embodiments may involve extracting indirect identifiers from the timestamps and events. The indirect identifiers may be properties of the data distribution, for example length of the time window, number of breaks in the data distribution, etc. Other elements of the data distribution can be categorized as indirect identifiers.

Further embodiments may involve treating events attached to the timestamps (e.g. ICD codes), when these are indirect identifiers in the following structure manner:

If the number of these events is lower than a threshold N (e.g. 5), then the ordered set of the explicit events represents the indirect identifier;

If the number of events is higher than said threshold N, the number of events becomes an indirect identifier. Events are not truncated from the dataset, nor are dummy ones added to the dataset.

Events in a specific category that are present in the dataset less than a threshold E will be generalized until they end-up in a category with the size higher than the threshold E.

The above thresholds N an E may be selected in view of the power of an attacker and the nature of the data.

The methods according to the invention may be implemented on a computer as a computer implemented method, or in dedicated hardware, or in a combination of both. Executable code for a method according to the invention may be stored on a computer program product. Examples of computer program products include memory devices such as a memory stick, optical storage devices such as an optical disc, integrated circuits, servers, online software, etc.

The computer program product in a non-transient form may comprise non-transitory program code means stored on a computer readable medium for performing a method according to the invention when said program product is executed on a computer. In an embodiment, the computer program comprises computer program code means adapted to perform all the steps or stages of a method according to the invention when the computer program is run on a computer. Preferably, the computer program is embodied on a computer readable medium. There is also provided a computer program product in a transient form downloadable from a network and/or stored in a volatile computer-readable memory and/or microprocessor-executable medium, the product comprising program code instructions for implementing a method as described above when executed on a computer.

Another aspect of the invention provides a method of making the computer program in a transient form available for downloading. This aspect is used when the computer program is uploaded into, e.g., Apple's App Store, Google's Play Store, or Microsoft's Windows Store, and when the computer program is available for downloading from such a store.

Further preferred embodiments of the devices and methods according to the invention are given in the appended claims, disclosure of which is incorporated herein by reference.

### BRIEF DESCRIPTION OF THE DRAWINGS

These and other aspects of the invention will be apparent from and elucidated further with reference to the embodiments described by way of example in the following description and with reference to the accompanying drawings, in which

FIG. 1 shows an example of longitudinal data,

FIG. 2 shows an example of a frequency table for the number of events,

FIG. 3 shows a schematic flow chart illustrating an embodiment of the method for anonymization of a set of patient data,

FIGS. 4a-4d show data distributions of time stamps in the longitudinal data,

FIG. 5 shows longitudinal data, indirect identifiers and equivalence classes,

FIG. 6a shows a computer readable medium, and

FIG. 6b shows in a schematic representation of a processor system.

The figures are purely diagrammatic and not drawn to scale. In the Figures, elements which correspond to elements already described may have the same reference numerals.

### DETAILED DESCRIPTION OF EMBODIMENTS

The present invention will be described with respect to particular embodiments and with reference to the figures, but the invention is not limited thereto, but only to the claims.

The term “individual” refers to a human subject. Said human subject may or may not be affected by or suffering from a disease to be studied. Hence, the terms “individual”, “person” and “patient” are synonymously used in the instant disclosure.

The expression “providing patient data” is understood that the patient data of at least one individual need to be obtained. However, the patient data of the at least one individual do not have to be obtained in direct association with the method or for performing the method. Typically the patient data of the at least one individual are obtained at a previous point or period of time, and are stored electronically in a suitable electronic storage device and/or database. For performing the method, the patient data can be retrieved from the storage device or database and utilized.

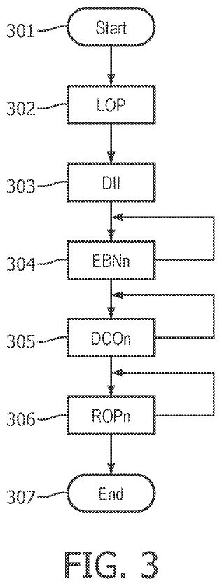

FIG. 3 shows a schematic flow chart illustrating an embodiment of the method for anonymization of a set of patient data. The set has longitudinal data from multiple patients. The method provides a predefined anonymity property, for example k-anonymity. According to the property a concatenation of all indirect identifiers of a patient enables identifying an outlying patient in the data set if there are less than a predefined value (e.g. k) patients having a same concatenation of indirect identifiers. The concatenation embodies the combined set of the values of all indirect identifiers. The set is considered to be potentially sufficient for recognizing an individual among the patients in the set. The longitudinal data in the data set at least includes events related to a disease or a treatment of a disease, and time stamps related to the events.

The method starts at node START 301, and step LOP 302 represents collecting and storing a set of longitudinal patient data of multiple individuals. Optionally, in the step LOP includes replacing time stamps representing dates by the time stamps representing intervals between the dates. Thereby, all time related data is made relative and cannot be matched to actual, individual dates and events.

Also, the method may include determining, across the data set, respective numbers of events in respective event categories regarding a respective disease or treatment. Rare events may potentially help an attacker to identify an individual. Then, any outlying event category is determined where the respective number of events is less than an event threshold (E). All events of the outlying respective event category are generalized until these events end-up in an event category where the respective number of events is higher than the threshold. For example, the threshold E may be 10.

In next step DII 303 indirect identifiers are determined, including at least one first indirect identifier representing a property of the data distribution of the time stamps and at least one second indirect identifier representing a number of events regarding a respective patient. In an embodiment, the first indirect identifier may be a length of a time window covering all time stamps from an individual, e.g. a total period in years.

In an embodiment, a first identifier may be a number of breaks in such a time window. A break represents a local minimum in the distribution of the events during the time window, indicative of a substantial period in the total time window without, or with relatively few, events. For example, if events for a patient are succeeding every day for one week, then nothing happens for one week and then again they start repeating every day, then the break is the week in the middle and therefore is called a local minimum. In a further embodiment, the method periods of a predetermined length in a sequence of events from an individual are determined. Then, a number of breaks in the periods is determined as the first indirect identifier, a break being a local minimum in the distribution of the events during the periods. Optionally, the method comprises determining, as a first indirect identifier, intervals of a predetermined length that have no events in respective sequences of events of respective patients. For example, as the second indirect identifier, a logarithmic function of the number of events regarding a respective individual may be used, while the value may be rounded to an integer.

Optionally, in a next step EBNn 304, repeatedly for all n patients in the data set, it is determined whether the number of events regarding a respective patient is below a number threshold (N). For example, N=5 and for any patient having 5 or more events the number of events is considered to be an indirect identifier. However, when the number of events is below N, the set of events regarding the respective patient is taken as a further indirect identifier. In an embodiment, the set of events is an ordered list of events. Optionally, when taking as the second indirect identifier the rounded logarithmic function base 10 of the number of events the value N, this function will be zero for 3 or less events, so N=4 coincides with the function round(log₁₀(x)).

In next step DCOn 305, repeatedly for all n patients in the data set, concatenations of all indirect identifiers are determined, which concatenations represent equivalence classes of potentially identifiable individuals. The respective concatenations comprise the above determined first indirect identifier and the second indirect identifier, an any further indirect identifiers. Optionally, various first, second and further indirect identifiers may be included in said concatenation, where such combination of indirect identifiers is considered to constitute a risk of identifying the individual.

Subsequently, in next step ROPn 306, repeatedly for all n patients in the data set, the patient data of each outlying patient is removed from the data set. An outlying patient is any patient for which there are less than a predefined value (e.g. k) patients in an equivalence class, e.g. having a same concatenation of indirect identifiers.

Finally, the now anonymized data set may be provided as output to be used for further data analysis, research or statistics. The method terminates at node END 307.

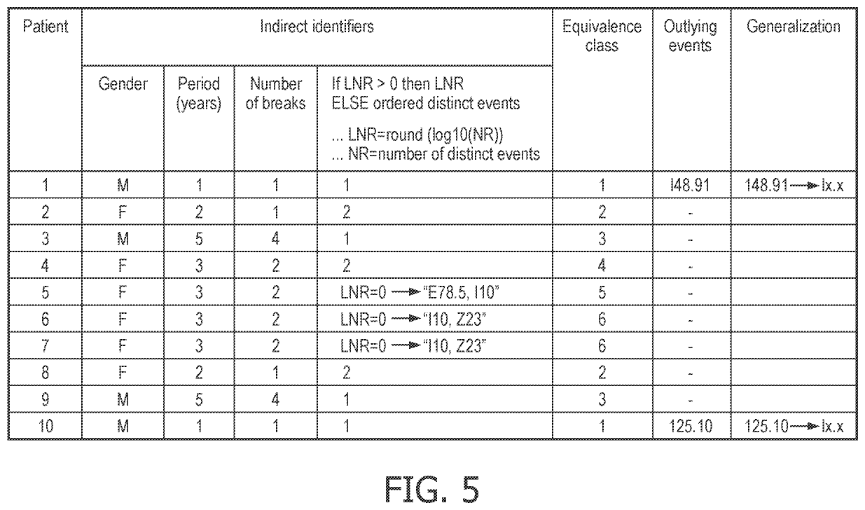

Various embodiments may be implemented as a software framework that de-identifies longitudinal data by shifting dates, generalizing outlying events and suppressing outlying patients as depicted in FIG. 5 and discussed later. In the de-identification process the first indirect identifiers are extracted from the timestamps, in particular from the distribution of the timestamps.

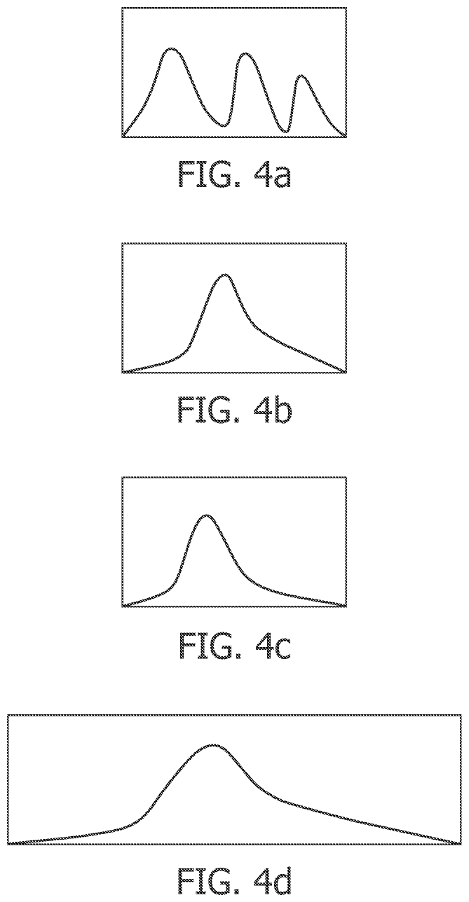

FIGS. 4a-4d show data distributions of time stamps in the longitudinal data. Each graph shows the number of events (y-axis) in time (x-axis).

FIG. 4a shows an example distribution in a time window of one year having two breaks.

FIG. 4b shows an example distribution in a time window of one year having zero breaks.

FIG. 4c shows a further example distribution in a time window of one year having zero breaks.

FIG. 4d shows an example distribution in a time window of two years having zero breaks.

Main indirect identifiers can be the length of the time window covered by all timestamps, furthermore other elements of the distribution of these timestamps, etc. The choice regarding these indirect identifiers depends on the assumed power of the attacker and the nature of the data. This choice may be done in a preparatory process based on statistical data. The evaluation may further assisted by a de-identification expert. For examples for persons with a rich medical history diseases, the shortest interval between consecutive timestamps may not be a difference maker, but intervals without events may be an indirect identifier. For example in FIG. 4 distributions 4b and 4c are alike, while 4a has more breaks and 4d contains events over a longer period of time.

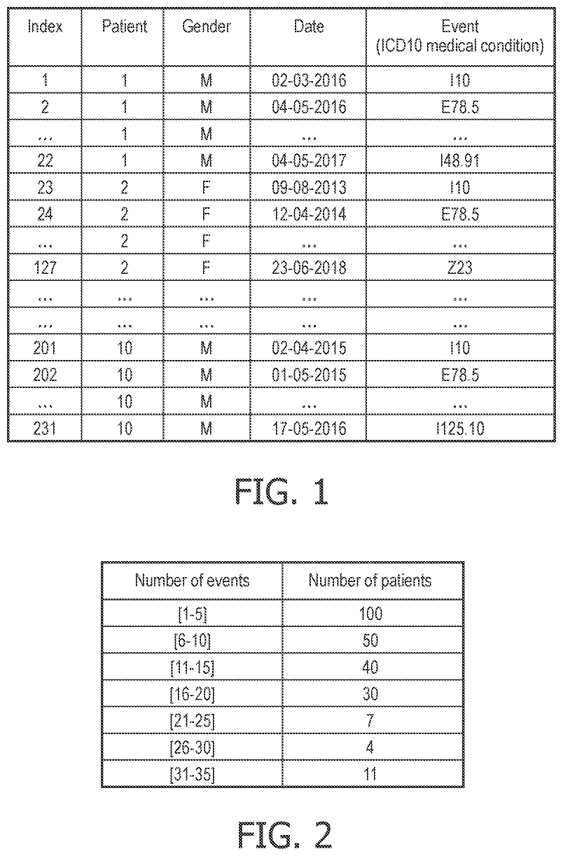

FIG. 5 shows longitudinal data, indirect identifiers and equivalence classes. The Figure shows how to make the longitudinal data depicted in FIG. 1 may be made 2-anonymous, where k=2 in the k-anonymity property. Firstly the “period” in years and the “number of breaks” are extracted from the timestamps distribution of the data. Then the “number of distinct events” and outlying events are extracted from the events of each patient longitudinal record. An adversary is very unlikely to know the exact number of events and therefore we applied the function round(log₁₀(x)) on the number of distinct events (x being the number of events). The choice of the function depends on the nature of the data and attacker knowledge, and may be automated. An attacker can usually differentiate only between a couple (e.g. 4-5) of categories for the values of one indirect identifier.

The number of categories is set in view of the power of an assumed attacker and the nature of the data. Once the number of categories is set, the belonging category of each value x may be set by means of normalization.

Normalizing the respective category between a minimum value (value_min) and a maximum value (value_max) can be done for example:
c=round(((x−value_min)/(value_max−value_min))*nr_categories)
   or, as exemplified above, using a logarithmic scale
   c=round(log L(x−value_min)), where L can be extracted from
   round(log L(value_max−value_min))=nr_categories.

Optionally, the rare events, which occur less than a threshold E in the total data set, are generalized for patients 1 and 10, where the respective disease code I48.91 is changed to Ix.x, and the respective disease code I25.10 is also changed to Ix.x. In the examples, the codes are ICD9 or ICD10 codes, e.g. diseases, procedures, as defined in [ICD]. In a further example, two codes needing generalization may have been I48.91 and I47.9. In that case the generalization may have been I4x.x.

Also, the longitudinal records with less than a threshold N, e.g. 4, distinct events have as an indirect identifier the ordered distinct events (e.g. patients 5, 6 and 7). Establishing the respective thresholds N and E may be done depending on the data set, e.g. by a de-identification expert. For example, the ceiling for the threshold N is around 20. The ceiling is used when the timestamps and events are the source of the only indirect identifiers. If more indirect identifiers are used, these are thresholds me be lowered.

After determining the indirect identifiers extracted from the longitudinal data, the next actions are perform for de-identifying the data. First, the values of all indirect identifiers are determined from the data set, while the set of values for each patient, also called a concatenation, is calculated. Then, all outlying patients are suppressed which are outliers because of their respective concatenation of indirect identifiers occurs less the k times in the data set. Removing such patients is not detrimental to the value of the data set, while traditional methods like generalizing dates is not advisable and may add noise in the data and risk affecting any research results. In the example, additionally, outlying events are generalized, as depicted in the column marked Generalization in FIG. 5. Also, dates may be converted into relative periods or dates may be shifted with a random number of days (e.g. between 50 and 100 years), different between patients, but which number of days is the same for the same patient.

The above methods may be applied in heath data analysis platform or similar platforms. It may also be used as a client application that interacts with a data-lake for making available (k-anonymous) longitudinal to its clients. Furthermore, the methods may be applied on any form of privacy preserving computation that results in a dataset that still contains personal information and any data export, e.g. for research.

In an embodiment, the method for anonymization of patient data may be used for performing medical research and can include bioinformatic means, e.g. by using software tools for an in silico analysis of biological queries using mathematical and statistical techniques to analyze and interpret biological data with respect to their relevance for the goal of the medical research. This embodiment typically requires use of genetic information of a plurality of individuals.

In another embodiment of the method for anonymization of patient data, the method may be used in diagnostics, wherein the genetic information of an individual is analyzed for the genetic disposition and/or occurrence of a specific disease or disorder of said individual.

The method may be applied to any disease, disorder or medical condition. A disease to be studied may be a specific disease that is chosen on purpose. In an embodiment, the disease to be studied is known to be a disease that is associated with a particular genotype. Examples of such diseases are cancers, immune system diseases, nervous system diseases, cardiovascular diseases, respiratory diseases, endocrine and metabolic diseases, digestive diseases, urinary system diseases, reproductive system diseases, musculoskeletal diseases, skin diseases, congenital disorders of metabolism, and other congenital disorders such as prostate cancer, diabetes, metabolic disorders, or psychiatric disorders.

Patient data not directly related to a disease to be studied may be anonymized by using techniques that are selected from the group consisting of statistical anonymization, encryption, and secure multiparty anonymization and computation.

These anonymization techniques allow analysis on the data, but this analysis is limited due to their properties. The statistical anonymization implies loss of information, but keeps the rest of the information in a human-readable shape. This allows analyses to be performed on the data, but the results are limited by the loss of information from the beginning. Encryption techniques do not lose information, but this information is not available. However, if there is ever any indication that the encryption information is necessary for research, a privacy officer is able to extend the core disease information by decrypting this set. Modern techniques like homomorphic encryption, multi-party computations and/or other operations on encrypted data may be used on the longitudinal data. In these situations the privacy-sensitive information will stay secret, while the result of these operations can be disclosed by the privacy officer. These techniques insert latency in the analysis and therefore are limiting the possible analyses that can be performed on the data.

In an embodiment, the anonymity property is selected from the group consisting of k-anonymity, l-diversity, t-closeness and δ-presence.

K-anonymity is a formal model of privacy created by Sweeney [4]. The goal is to make each record indistinguishable from a defined number (k) of other records if attempts are made to identify the data. A set of data is k-anonymized if, for any data record with a given set of attributes, there are at least k−1 other records that match those.

L-diversity improves anonymization beyond what k-anonymity provides. The difference between the two is that while k-anonymity requires each combination of quasi identifiers to have k entries, l-diversity requires that there are l different sensitive values for each combination of quasi identifiers, see [6].

T-closeness requires that the distribution of a sensitive attribute in any equivalence class is close to the distribution of the attribute in the overall table (i.e., the distance between the two distributions should be no more than a threshold T), see [7]. L-diversity requirement ensures “diversity” of sensitive values in each group, but it does not take into account the semantically closeness of these values. This is done by t-closeness.

δ-presence is a metric to evaluate the risk of identifying an individual in a table based on generalization of publicly known data. δ-presence is a good metric for datasets where “knowing an individual is in the database poses” a privacy risk, see [8].

The anonymization techniques may comprise “searchable encryption”, “homomorphic encryption”, and “secure multiparty computation”, which have the advantage that decryption of the encrypted data is not actually necessary, but it is feasible to perform data processing in the encrypted domain. The main difference between these techniques is the choice of trade-offs they make. Searchable encryption limits the processing to a simple keyword match. Fully homomorphic encryption can do any kind of processing, but has extremely big ciphertext sizes and is computationally very intensive. Multiparty computation scales better, but requires non-colluding computers to work together to do the processing.

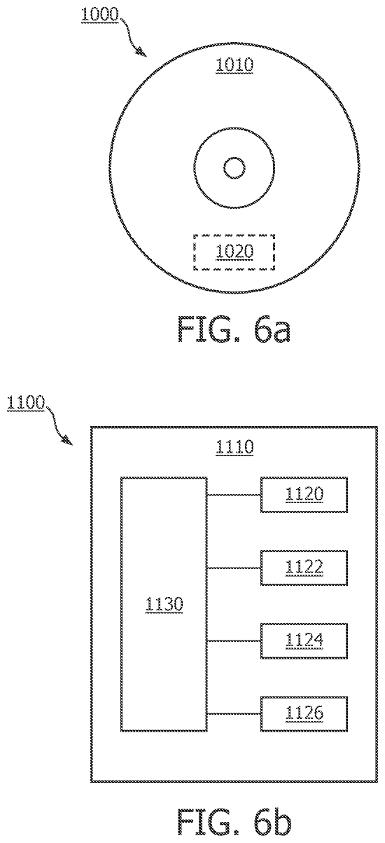

In an embodiment, the method as described in FIG. 3 may be implemented in a system 1100 as depicted in FIG. 6b, discussed later, e.g. on a computer as a computer implemented method, as dedicated hardware, or as a combination of both. As also illustrated in FIG. 6a, instructions for the computer, e.g., executable code 1020, may be stored on a computer readable medium 1000, e.g., in the form of a series of machine readable physical marks and/or as a series of elements having different electrical, e.g., magnetic, or optical properties or values. The executable code may be stored in a transitory or non-transitory manner. Examples of computer readable mediums include memory devices, optical storage devices, integrated circuits, servers, online software, etc. The Figure shows an optical disc 1010.

It will be appreciated that the invention applies to computer programs, particularly computer programs on or in a carrier, adapted to put the invention into practice. The program may be in the form of a source code, an object code, a code intermediate source and an object code such as in a partially compiled form, or in any other form suitable for use in the implementation of the method according to the invention. It will also be appreciated that such a program may have many different architectural designs. For example, a program code implementing the functionality of the method or system according to the invention may be sub-divided into one or more sub-routines. Many different ways of distributing the functionality among these sub-routines will be apparent to the skilled person. The sub-routines may be stored together in one executable file to form a self-contained program. Such an executable file may comprise computer-executable instructions, for example, processor instructions and/or interpreter instructions (e.g. Java interpreter instructions). Alternatively, one or more or all of the sub-routines may be stored in at least one external library file and linked with a main program either statically or dynamically, e.g. at run-time. The main program contains at least one call to at least one of the sub-routines. The sub-routines may also comprise function calls to each other. An embodiment relating to a computer program product comprises computer-executable instructions corresponding to each processing stage of at least one of the methods set forth herein. These instructions may be sub-divided into sub-routines and/or stored in one or more files that may be linked statically or dynamically. Another embodiment relating to a computer program product comprises computer-executable instructions corresponding to each means of at least one of the systems and/or products set forth herein. These instructions may be sub-divided into sub-routines and/or stored in one or more files that may be linked statically or dynamically.

The carrier of a computer program may be any entity or device capable of carrying the program. For example, the carrier may include a data storage, such as a ROM, for example, a CD ROM or a semiconductor ROM, or a magnetic recording medium, for example, a hard disk. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

FIG. 6a shows a computer readable medium 1000 having a writable part 1010 comprising a computer program 1020, the computer program 1020 comprising instructions for causing a processor system to perform one or more of the above methods and processes in the system as described with reference to FIGS. 1-4. The computer program 1020 may be embodied on the computer readable medium 1000 as physical marks or by means of magnetization of the computer readable medium 1000. However, any other suitable embodiment is conceivable as well. Furthermore, it will be appreciated that, although the computer readable medium 1000 is shown here as an optical disc, the computer readable medium 1000 may be any suitable computer readable medium, such as a hard disk, solid state memory, flash memory, etc., and may be non-recordable or recordable. The computer program 1020 comprises instructions for causing a processor system to perform said methods.

FIG. 6b shows in a schematic representation of a processor system 1100 according to an embodiment of the devices or methods as described with reference to FIGS. 1-5. The processor system may comprise a circuit 1110, for example one or more integrated circuits. The architecture of the circuit 1110 is schematically shown in the Figure. Circuit 1110 comprises a processing unit 1120, e.g., a CPU, for running computer program components to execute a method according to an embodiment and/or implement its modules or units. Circuit 1110 comprises a memory 1122 for storing programming code, data, etc. Part of memory 1122 may be read-only. Circuit 1110 may comprise a data interface 1126, comprising, e.g., an antenna, a transceiver for internet, connectors or both, and the like. Circuit 1110 may comprise a dedicated integrated circuit 1124 for performing part or all of the processing defined in the method. Processor 1120, memory 1122, dedicated IC 1124 and communication element 1126 may be connected to each other via an interconnect 1130, say a bus. The processor system 1110 may be arranged for wired and/or wireless communication, using connectors and/or antennas, respectively.

The system 1100 is configured to anonymizing patient data as described with the above methods, e.g. elucidated with reference to FIG. 3. The system comprises a data interface 1126 configured to access patient data of multiple individuals. The data interface may be in communicative with database on a local storage unit or on a server. The data interface may be connected to an external repository, such as a suitable electronic storage device and/or database, which comprises the patient data. Alternatively, the patient data or a database may be accessed from an internal data storage of the system 1122. In general, the data interface may take various forms, such as a network interface to a local or wide area network, e.g., the Internet, a storage interface to an internal or external data storage, etc.

Furthermore, the system 1100 may have a user input interface configured to receive user input commands from a user input device to enable the user to provide user input, such as choose or define a particular disease, disorder or medical condition for subsequently determining a subset of patient data being related to said disease, disorder or medical condition. The user input device may take various forms, including but not limited to a computer mouse, touch screen, keyboard, etc.

It will be appreciated that, for clarity, the above description describes embodiments of the invention with reference to different functional units and processors. However, it will be apparent that any suitable distribution of functionality between different functional units or processors may be used without deviating from the invention. For example, functionality illustrated to be performed by separate units, processors or controllers may be performed by the same processor or controllers. Hence, references to specific functional units are only to be seen as references to suitable means for providing the described functionality rather than indicative of a strict logical or physical structure or organization. The invention can be implemented in any suitable form including hardware, software, firmware or any combination of these.

According to a further aspect, the invention concerns the use of the method and/or the computer program product in research and/or in diagnosis. In an embodiment, the method and/or computer program product is used in bioinformatics research. The use of the method, system and/or computer program product in bioinformatics research comprises acquisition the patient data of a plurality of individuals. Examples of research fields are genomics, genetics, transcriptomics, proteomics and systems biology.

In an alternative embodiment, the method, system and/or computer program product may be used in diagnosis, wherein the patient data of an individual are utilized to analyze whether the individual is affected by a specific disease or at risk of getting said disease or being affected by said disease. The individuals are sure that their patient data are properly anonymized.

Where an indefinite or definite article is used when referring to a singular noun, e.g. “a”, “an”, “the”, this includes a plural of that noun unless something else is specifically stated. Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Moreover, the terms top, bottom, over, under, beyond and the like in the description and in the claims are used for descriptive purposes and not necessarily for describing relative positions. It is to be understood that the terms so used are interchangeable under appropriate circumstances and that the embodiments of the invention described herein are capable of operation in other orientations than described or illustrated herein. It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

### REFERENCES

ICD http://www.who.int/classifications/icd/en/
CPT https://www.medicalbillingandcoding.org/intro-to-cpt/
   The following documents are incorporated by reference herein for all purposes.
[1] GPDR—Council of European Union. Regulation (eu) 2016/679 of the European parliament and of the council of 27 Apr. 2016 on the protection of natural persons with regard to the processing of personal data and on the free movement of such data, and repealing directive 95/46/ec (general data protection regulation) (text with eea relevance), April 2016.
[2] Khaled El Emam and Luk Arbuckle: Anonymizing Health Data: Case Studies and Methods to Get You Started. O'Reilly Media, Inc., 1st edition, 2013.
[3] Khaled El Emam, Luk Arbuckle, Gunes Koru, Benjamin Eze, Lisa Gaudette, Emilio Neri, Sean Rose, Jeremy Howard, and Jonathan Gluck: De-identification methods for open health data: The case of the heritage health prize claims dataset. Journal of Med Internet Res, 14(1):e33, February 2012.
[4] Pierangela Samarati and Latanya Sweeney: Generalizing data to provide anonymity when disclosing information (Extended Abstract). In Proceedings of the Seventeenth ACM SIGACT-SIGMOD-SIGART Symposium on Principles of Database Systems, Jun. 1-3, 1998, Seattle, Wash., USA, page 188, 1998.
[5] HIPAA—The health insurance portability and accountability act; U.S. Dept. of Labor, Employee Benefits Security Administration, 2004.
[6] J. Sedayao, “Enhancing Cloud Security Using Data Anonymization,” June 2012. Available from: http://www.intel.nl/content/dam/www/public/us/en/documents/best-practices/enhancing-cloud-security-using-data-anonymization.pdf.
[7] N. Li, T. Li and S. Venkatasubramanian, “t-Closeness: Privacy Beyond k-Anonymity and l-Diversity,” in Data Engineering, 2007. ICDE 2007. IEEE 23rd International Conference on, 2007.
[8] M. E. Nergiz, M. Atzori and C. Clifton, “Hiding the Presence of Individuals from Shared Databases,” in Proceedings of the 2007 ACM SIGMOD International Conference on Management of Data, Beijing, China, 2007.

1. A computer-implemented method for anonymization of a data set of patient data from multiple patients for providing a predefined anonymity property,
wherein the property defines that a concatenation of all indirect identifiers of a patient enables identifying an outlying patient in the data set if there are less than a predefined value (k) patients having a same concatenation of indirect identifiers, the patient data comprising
events related to a disease or a treatment of a disease,
time stamps related to the events;
the method comprising the steps of:
determining at least one first indirect identifier representing a property of the data distribution of the time stamps,
determining at least one second indirect identifier representing a number of events regarding a respective patient,
determining, for all patients in the data set, the respective concatenations comprising the first indirect identifier and the second indirect identifier,
removing the patient data of each outlying patient from the data set.

2. The method according to claim 1, wherein the method comprises
determining whether the number of events regarding a respective patient is below a number threshold (N), and, if so,
determining, as a third indirect identifier, the set of events regarding the respective patient.

3. The method according to claim 1, wherein the set of events is an ordered list of events.

4. The method according to claim 1, wherein the first indirect identifier represents a length of a time window covering all time stamps from an individual.

5. The method according to claim 4, wherein the method comprises
determining a number of breaks in the time window as a further indirect identifier, a break being a local minimum in the distribution of the events during the time window.

6. The method according to claim 1, wherein the method comprises
determining periods of a predetermined length in a sequence of events from an individual, and
determining a number of breaks in the periods as the first indirect identifier, a break being a local minimum in the distribution of the events during the periods.

7. The method according to claim 1, wherein the method comprises
determining, as the first indirect identifier, intervals of a predetermined length that have no events in respective sequences of events of respective patients.

8. The method according to claim 1, wherein the method comprises,
determining of a number of categories (nr_categories) for values (x) of a respective indirect identifier that attacker may differentiate,
normalizing to a normalized value (c) the respective category between a minimum value (value_min) and a maximum value (value_max):
c=round(((x−value_min)/(value_max−value_min))*nr_categories)

9. The method according to claim 1, wherein the method comprises,
determining of a number of categories (nr_categories) for values (x) up to a maximum value (value_max) of a respective indirect identifier that attacker may differentiate,
normalizing to a normalized value (c) the respective category:
c=round(log L(x)), wherein L is extracted from round(log L(value_max))=nr_categories.

10. The method according to claim 1, wherein the method comprises
using as the second indirect identifier a logarithmic function of the number of events regarding a respective individual.

11. The method according to claim 1, wherein the method comprises
determining, across the data set, respective numbers of events in respective event categories regarding a respective disease or treatment,
determining at least one outlying event category where the respective number of events is less than an event threshold (E), and
generalizing the outlying respective event category until the events end-up in an event category where the respective number of events is higher than the threshold.

12. The method according to claim 1, wherein the method comprises replacing time stamps representing dates by the time stamps representing intervals between the dates.

13. A computer program product for anonymization of a data set of patient data from multiple patients for providing a predefined anonymity property, the computer program product comprising instructions which when carried out on a computer cause the computer to perform a method as claimed in claim 1.

14. A system for anonymization of a data set of patient data from multiple patients for providing a predefined anonymity property,
wherein the property defines that a concatenation of all indirect identifiers of a patient enables identifying an outlying patient in the data set if there are less than a predefined value (k) patients having a same concatenation of indirect identifiers,
the patient data comprising
events related to a disease or a treatment of a disease,
time stamps related to the events;
said system comprising:
a data interface configured to receive patient data of at least one patient, and a processor arranged to
determine at least one first indirect identifier representing a property of the data distribution of the time stamps,
determine at least one second indirect identifier representing a number of events regarding a respective patient,
determine, for all patients in the data set, the respective concatenations comprising the first indirect identifier and the second indirect identifier,
remove the patient data of each outlying patient from the data set.

15. Use of the method according to claim 1, the computer program product and/or the system in one selected from the group consisting of genomics, genetics, bioinformatics research, transcriptomics, proteomics and systems biology or diagnosis.
